(19) United States
(12) Patent Application Publication
(10) Pub. No.: US 2019/0169562 A1
(43) Pub. Date: Jun. 6, 2019

(54) SCREENING OF IMMUNO-MODULATORY THERAPIES

(71) Applicant: zPredicta, Inc., San Jose, CA (US)

(72) Inventors: Julia Kirshner, San Jose, CA (US); Mukti Rajen Parikh, San Jose, CA (US)

(73) Assignee: zPredicta, Inc., San Jose, CA (US)

(21) Appl. No.: 16/321,277

(22) PCT Filed: Jul. 31, 2017
(86) PCT No.: PCT/US17/44736

§ 371 Date: Jan. 28, 2019

Related U.S. Application Data
(60) Provisional application No. 62/368,979, filed on Jul. 29, 2016.

(51) Int. Cl. C12M 1/34; G01N 33/50

(52) U.S. Cl. CPC C12M 41/46; G01N 33/5011; G01N 33/505; G01N 33/5029

(57) ABSTRACT

Embodiments provide methods of preparing a platform, which entails, among others, contacting a) a culture comprising a medium or growth matrix comprising an immune cell, with b) a biological matrix mimetic comprising a target cell such a cancer cell, and allowing, under suitable conditions, the immune cell to migrate to the biological matrix mimetic.

### CROSS REFERENCE TO RELATED APPLICATIONS

This application claims the benefit under 35 U.S.C. § 119(e) of the U.S. Provisional Application Ser. No. 62/368,979, filed Jul. 29, 2016, the content of which is hereby incorporated by reference in its entirety.

### BACKGROUND

Immunotherapy is the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Immunomodulatory regimens often have fewer side effects than existing drugs, including less potential for creating resistance in microbial disease.

Cell-based immunotherapies are effective for some cancers. Immune effector cells such as lymphocytes, macrophages, dendritic cells, natural killer cells (NK Cell), cytotoxic T lymphocytes (CTL), etc., work together to defend the body against cancer by targeting abnormal antigens expressed on the surface of tumor cells.

Therapies such as granulocyte colony-stimulating factor (G-CSF), interferons, imiquimod and cellular membrane fractions from bacteria have been used clinically. Others including IL-2, IL-7, IL-12, various chemokines, synthetic cytosine phosphate-guanosine (CpG) oligodeoxynucleotides and glucans are involved in clinical and preclinical studies.

### SUMMARY

The present disclosure provides methods and compositions for screening for immune-modulatory agents useful for treating diseases. In one embodiment, a culture is provided that has a medium or growth matrix containing an immune cell. Also provided is a biological matrix mimetic containing a target cell. The biological matrix mimetic mimics an organ or tissue. Under suitable conditions, the immune cell migrates to get in contact with the target cells and exhibits impact over the target cell. Such a method and platform can be useful for assessing or screening conditions or agents that can be used to modulate the immune cell to target cell interaction. Also provided, in some embodiments, are cultures and biological matrix mimetics or their combinations for such methods.

One embodiment of the present disclosure provides a method of preparing a platform, comprising: contacting a) a culture comprising a medium or growth matrix and an immune cell in the medium or growth matrix, with b) a biological matrix mimetic comprising a target cell, and allowing, under suitable conditions, the immune cell to migrate to the biological matrix mimetic. In some embodiments, the method further comprises preparing the biological matrix mimetic by adding the target cell to a biological matrix mixture and polymerizing the biological matrix mixture.

In some embodiments, the biological matrix mixture comprises collagen IV (CIV), laminin (LN), fibronectin (FN), collagen I (CI), and hyaluronic acid (HA), wherein the CIV, LN, FN, VI and HA are present at a ratio of about (1.5-3):(1.5-3):(2-3):1:1. In some embodiments, the CIV, LN, FN, VI and HA are present at a ratio of about 2:2:2.5:1:1. In some embodiments, the biological matrix mimetic is overlaid on a surface coated with an endosteum mimetic. In some embodiments, the endosteum mimetic comprises fibronectin (FN) and collagen I (CI), and the FN and CI are present at a ratio of about (0.75-3.5):1.

In some embodiments, the immune cell is a T cell, B cell, a natural killer cell, a macrophage, a neutrophil, an eosinophil, or a dendritic cell. In some embodiments, the T cell comprises a chimeric antigen receptor (CAR) or the T cell is activated by an immunomodulatory agent. In some embodiments, the suitable conditions allow the CAR to bind the target cell.

In some embodiments, the culture or the biological matrix mimetic further comprises a candidate immune-modulatory agent. In some embodiments, the immune-modulatory agent targets a disease selected from the group consisting of an inflammatory disease and cancer. In some embodiments, the immune-modulatory agent is a checkpoint inhibitor. In some embodiments, the checkpoint inhibitor inhibits a molecule selected from the group consisting of A2AR, B7-H3, B7-H4, BTLA, CTLA-4, IDO, KIR, LAG3, PD-1, TIM-3 and VISTA.

In some embodiments, the method further comprises adding an immune activation agent. In some embodiments, the addition of the immune activation agent is before or after the immune cell migrates to the biological matrix mimetic. In some embodiments, the immune activation agent is a chemokine or a cytokine.

In some embodiments, the culture contacts with the biological matrix mimetic through a fluid, wherein the fluid has a higher serum concentration than the culture. In some embodiments, the fluid contains at least 8% serum or serum substitute. In some embodiments, the fluid contains at least 15% serum or serum substitute. In some embodiments, the culture contains less than 5% serum or serum substitute. In some embodiments, the fluid has serum concentration that is at least twice of that in the culture. In some embodiments, the medium or growth matrix is derived from a healthy vertebrate.

In some embodiments, the biological matrix mimetic comprises organ-specific matrix. In some embodiments, the organ-specific matrix simulates the adrenal gland, bone marrow, brain, liver, lung tissue, lymph node, ovary, peritoneum, skin, spleen, connective tissue, bone, vascular structure, or articular joint. In some embodiments, the biological matrix mimetic comprises collagens 1-14 or fragments thereof, elastin, laminin, fibronectin, hyaluronic acid or related hyaluronans, lecticans, or other glycosaminoglycans, chondroitins, dermatans, or related extracellular matrix or glycocalyx components or combinations thereof.

In some embodiments, the biological matrix mimetic comprises a solid tumor of the bladder, bone, bone marrow, brain, breast, cervix, colon, endometrium, esophagus, kidney, liver, lung, skin, ovary, pancreas, prostate, stomach, testicle, thyroid, or uterus, endothelial cells, smooth muscle cells, pericytes, scars, fibrotic tissue, surgical adhesion tissue or hyperproliferative bone lesions. In some embodiments, the target cell is a tumor cell embedded in the biological matrix mimetic.

In some embodiments, the culture comprises collagen I, collagen II, collagen III, collagen IV, collagen-V, elastin, laminin, fibronectin, hyaluronic acid, lecticans, or combinations thereof. In some embodiments, the suitable conditions comprise a temperature of from about 30° C. to about 45° C.

In some embodiments, the method further comprises observing an impact of the immune cell on the viability or proliferation of the target cell.

In another embodiment, provided is a platform, comprising: a) a culture comprising a medium or growth matrix and an immune cell in the medium or growth matrix; and b) a biological matrix mimetic comprising a target cell, wherein the culture and the biological matrix mimetic are in sufficient contact to allow the immune cell to migrate to the biological matrix mimetic.

In some embodiments, the biological matrix mimetic is prepared by adding the target cell to a biological matrix mixture and polymerizing the biological matrix mixture. In some embodiments, the biological matrix mixture comprises collagen IV (CIV), laminin (LN), fibronectin (FN), collagen I (CI), and hyaluronic acid (HA), wherein the CIV, LN, FN, VI and HA are present at a ratio of about (1.5-3):(1.5-3):(2-3):1:1. In some embodiments, the CIV, LN, FN, VI and HA are present at a ratio of about 2:2:2.5:1:1.

In some embodiments, the biological matrix mimetic is overlaid on a surface coated with an endosteum mimetic. In some embodiments, the endosteum mimetic comprises fibronectin (FN) and collagen I (CI), and the FN and CI are present at a ratio of about (0.75-3.5):1. In some embodiments, the immune cell is a T cell, B cell, a natural killer cell, a macrophage, a neutrophil, an eosinophil, or a dendritic cell. In some embodiments, the T cell comprises a chimeric antigen receptor (CAR) or the T cell is In some embodiments, the target cell is a tumor cell.

### BRIEF DESCRIPTION OF THE DRAWINGS

Certain features of various embodiments of the present technology are set forth with particularity in the appended claims. A better understanding of the features and advantages of the technology will be obtained by reference to the following detailed description that sets forth illustrative embodiments, in which the principles of the technology are utilized, and the accompanying drawings of which:

FIG. 1 illustrates a process of preparing a reconstructed bone and testing immuno-modulatory agents and/or cells.

FIG. 2 is an example flow chart for setting up and using reconstructed organs.

FIG. 3 shows the results of an investigational antibody-drug conjugate used against multiple myeloma cells cultured in standard 2D culture (lower line) or in a reconstructed bone (r-Bone) (upper line).

FIG. 4 shows the results of testing bone marrow aspirated from patients with multiple myeloma (n=10) cultured in r-Bone system and treated with pomalidomide.

FIG. 5 illustrates the procedure used in Example 5.

FIGS. 6A and 6B show the results from the procedure of FIG. 5. When added to the r-Bone culture of tumor cells as prepared in FIG. 5, target-specific CAR-T cells were found interacting with, and ultimately, killing tumor spheroids.

### DETAILED DESCRIPTION

In the following detailed description, reference is made to the accompanying drawings, which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, over/under, back/front, and top/bottom. Such descriptions are merely used to facilitate the discussion and are not intended to restrict the application of embodiments.

Reconstructed Bone Marrow for Screening for Immune-Modulatory Agents and Cells

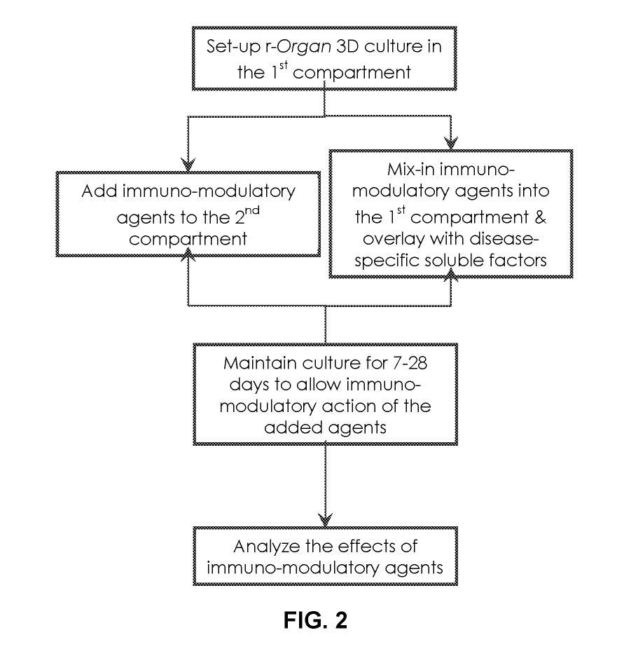

The present disclosure, in some embodiments, provides systems and methods for testing or evaluating candidate immuno-modulatory agents. As illustrated in FIG. 2, a target cell can be placed in a biological matrix mimetic (referred to as a reconstructed organ or rOrgan) in a first compartment. An immune cell, optionally with an immuno-modulatory agent, is placed in a culture medium or a growth medium. The culture can be in a separate compartment that is made in contact with the first compartment. The culture can also be placed on top or adjacent to the biological matrix mimetic.

Optionally, disease-specific soluble factors can be added to assist the testing. The immune cell and the target cell are cultured under suitable conditions to make in contact with each other so that the immune cell can exhibit activity over the target cell, optionally under the influence of the immuno-modulatory agent, thereby testing the immuno-modulatory activity of the immune cell and/or the immuno-modulatory agent.

The methods can employ organ-specific extracellular matrices and disease-specific soluble factors, depending on the type of cells and diseases. The candidate immuno-modulatory agents can be small molecules, antibodies, biologics, cells, any other agent with immuno-modulatory properties, chemoattractive agents.

The present technology, in some embodiments identifies immuno-oncology agents that have the capacity to activate the immune system against cancer. The technology can allow testing of immuno-modulatory agents under native conditions of human physiology in an organ and disease-specific manner.

The term “growth matrix” or “primary site growth matrix” refers to a matrix to mimic a tissue such as an organ. The tissue may be selected from a variety of sites, including bladder, bone, brain, breast, cervix, colon, esophagus, kidney, liver, lung, skin, ovary, pancreas, prostate, stomach, uterus, testicles, thyroid, and the like. In some embodiments, this matrix comprises a cell culture medium comprising RPMI-1640 with L-glutamine (or other growth medium) and about 1% horse serum. The matrix may also include an antimicrobial, antibiotic, and/or antifungal substance. For example, in some embodiments illustrated herein, growth medium comprises RPMI-1640 with L-glutamine, 20% fetal bovine serum (FBS), 6.2×10⁻⁴M CaCl₂, 1×10⁻⁶M sodium succinate, and 1×10⁻⁶M hydrocortisone and 1% penicillin/streptomycin.

Additional components may include basement membrane (BM), collagen (defined below as CI-V for collagen I, collagen II, collagen III, collagen IV, collagen V), fibronectin (FN), laminin (LN), hyaluronic acid (HA), elastin, lecticans, and the like.

Exemplary growth matrices are presented in Table A below. Also provided are representative concentrations and ranges of appropriate concentrations.

TABLE A
Growth Matrix

| Primary Tumor | | Matrix | |
|---|---|---|---|
| Site-Organ | Matrix | concentration | Concentration range |
| Bladder | LN; FN; CI; CIII; | 1:2:4:1:1 | (1-2):(1-2):(2-6): |
| | elastin | | (0-1):(0-1) |
| Brain | HA; lecticans | 5:1 | (2-10):1 |
| Breast | CIV; LN | 1:1 | (0.5-2):1 |
| Cervical | HA; CI; CIII | 5:1:1 | (2-10):(1-5):(0-1) |
| Colon | CIV; LN; FN | 1:1:1 | (1-2):(1-2):(1-3) |
| Esophagus | LN; FN; CI; CIII; | 1:2:4:1:1 | (1-2):(1-2):(2-6): |
| | elastin | | (0-1):(0-1) |
| Kidney | FN; CI; CIII | 2:3:1 | (1-2):(1-5):(0-1) |
| Liver | CIV; LN; FN; HA | 2:2:2.5:1 | (1-3):(1-3):(2-3):(1-3) |
| | (BM) | | |
| | CI; CIII; CV | 5:1:1 | (5-10):1:1 |
| Lung | CIV; LN (BM) | 1:1 | (0.5-2):1 |
| | CIV; LN; FN | 2:1 | (2-4):(1-3) |
| Melanoma | CIV; LN | 1 | (0.5-2): 1 |
| Ovarian | LN; FN; HA; CI; | 1:2:3:2:1 | (1-2):(1-2):(1-4): |
| | CIII | | (1-4):(0-1) |
| Pancreas | Collagen I | 1 | n/a |
| Prostate | CIV; LN | 1 | (0.5-2):1 |
| Sarcoma | CIV; LN; FN; HA; | 2:2:2:2:2 | (1.5-3):(1.5-3): |
| | CI¹/CI²/ CIII³ | | (1-3):(1-4):(1-2) |
| Stomach | CIV; LN; FN; HA | 2:2:2.5:1 | (1-3):(1-3):(2-3):(1-3) |
| Testicular | (1.5-3) | (1.5-3) | (1.5-3) |
| Thyroid | CIV; LN; FN | 1:1:1 | (1-2):(1-2):(1-3) |

¹osteosarcoma; ²myosarcoma; ³chondrosarcoma

For the purposes of describing embodiments, the phrases “biological matrix mimetic,” “reconstructed organ matrix”, “organ-specific matrix”, or “extracellular matrix” refer to any substance, solution, mixture, including a commercially available product, that is designed, produced, or used to mimic or approximate in vitro one or more biological matrices such as, for example, an extracellular matrix, an intracellular matrix, a basement membrane, and/or a structure of a connective tissue. This matrix mimics the site of the metastasis or secondary site. Matrices may be selected based on the secondary site or metastatic site. Additional components may include basement membrane (BM), collagen (defined below as CI-V for collagen I, collagen II, collagen III, collagen IV, collagen V), fibronectin (FN), laminin (LN), hyaluronic acid (HA) or related hyaluronans, elastin, lecticans, or other glycosaminoglycans, chondroitins, dermatans, or related extracellular matrix or glycocalyx components or combinations thereof, and the like. Representative matrices and concentrations are shown below in Table B.

TABLE B
Biological Matrix Mimetic

| | | Matrix | Concentration |
|---|---|---|---|
| Organ | Matrix | concentration | range |
| Adrenal gland | CIV; LN; FN; CI | 2:2:2.5:1 | (1.5-3):(1.5-3): |
| | | | (2-4):1 |
| Bone | FN; CI (endosteum) | 1:1 | (0.75-3.5):1 |
| | CIV; LN; FN; CI; | 2:2:2.5:1:1 | (1.5-3):(1.5-3): |
| | HA | | (2-3):1:1 |
| Brain | HA; lecticans | 5:1 | (2-10):1 |
| Liver | CIV; LN; FN; HA | 2:2:2.5:1 | (1-3):(1-3): |
| | (BM) | | (2-3):(1-3) |
| | CI; CIII; CV | 5:1:1 | (5-10):1:1 |
| Lung | CIV; LN (BM) | 1:1 | (0.5-2):1 |
| | CIV; LN; FN | 1:1:1 | (1-2):(1-2):(1-3) |
| Lymph node | CIV; LN; FN; CI; | 2:2:2.5:1:1 | (1.5-3):(1.5-3): |
| | CIII | | (2-4):(1-5):(0-1) |
| Ovary | CIV; LN | 1:1 | (0.5-2):1 |
| Peritoneum | CIV; LN; CI; FN | 2:2:2.5:1 | (1-3):(1-3): |
| | | | (2-4):1 |
| Skin | CIV; LN; FN (BM) | 1:1:1 | (1-2):(1-2):(1-3) |
| | CI; CIII | 3:1 | (1-6):(0:3) |
| Spleen | CIV; LN; HA (BM) | 2:2:1 | (1-3):(1-3):(1-10) |
| | FN; CI | 2:1 | (2-4):1 |

A biological matrix mimetic, in some embodiments, is a polymerized matrix which can be prepared from a “biological matrix mixture” through polymerization. Example ingredients in the biological matrix mixtures are as shown in Table B.

In some embodiments, a culture contacts with a biological matrix mimetic through a fluid, wherein the fluid has a higher serum concentration than the culture. As well known in the art, serum is the blood component after blood cells and clotting factors are removed and is the blood plasma not including the fibrinogens. Serum includes all proteins not used in blood clotting and all the electrolytes, antibodies, antigens, hormones, and any exogenous substances. Natural serum can be isolated from blood of vertebrates, either healthy or having cancer.

As used herein the term “fluid derived from a vertebrate” may include, but is not limited to peritoneal fluid, ascites fluid, cerebrospinal fluid, whole blood, plasma, or serum from blood or bone marrow, lymph and/or synovial fluid, tears, urine, saliva or any other gastrointestinal fluids from any vertebrate animal (including but not limited to humans, non-human primates, rats, mice, rabbits, pigs, dogs, and others). The vertebrate may be healthy, may have cancer or a premalignant syndrome. In one embodiment where the vertebrate does not have cancer and is healthy, tumor cells from the same source or different source may be added to the system. In embodiment where the vertebrate has cancer, this fluid may include plasma, serum, peritoneal fluid, ascites fluid, cerebrospinal fluid, blood, lymph and/or synovial fluid from any vertebrate animal (including but not limited to humans, non-human primates, rats, mice, rabbits, pigs, dogs, and others) with any form of cancer, including but not limited to solid tumors, cancers of bone, soft tissue, muscle, skin and/or blood. For the purposes of describing embodiments, the phrases “healthy vertebrate”, “normal vertebrate”, or “disease-free vertebrate” are used interchangeably and describe a vertebrate animal that is free of disease condition or pathology.

Synthetic, substitute or replacement serum (collectively referred to as “substitute serum”) can also be made and is commercially available. Substitute serums typically include most or all major serum proteins found in vertebrates. The major serum proteins include, for instance, albumins, globlulins, and regulatory proteins. More specific examples include, without limitation, prealbumin, alpha 1 antitrypsin, alpha 1 acid glycoprotein, alpha 1 fetoprotein, alpha2-macroglobulin, gamma globulins, beta-2-microglobulin, haptoglobin, ceruloplasmin, Complement component 3, Complement component 4, C-reactive protein (CRP), lipoproteins (chylomicrons, VLDL, LDL, HDL), transferrin, Mannan-binding lectin, and mannose-binding protein.

In some aspects, the serum or substitute serum concentration in the secondary site growth medium is at least 8%, 9% 10%, 12%, 15%, 18%, 20% or 25%. In some aspects, the serum or substitute serum concentration in the fluid of the first component is less than 6%, 5.5%, 5%, 4.5%, 4%, 3%, 2%, or 1.5%. In some aspects, the serum or substitute serum concentration in the secondary site growth medium is at least 50% higher than, 80% higher than, or is two times, three times, four times or five times the serum or substitute serum concentration in the fluid of the first component.

Additional components may be coupled, e.g., an incubator, microscope, pump, etc., to the cell culture assembly. The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

The cell culture assembly may employ tissue culture vessels or cell culture vessels. For the purposes of describing embodiments, the phrases “tissue culture vessel” and “cell culture vessel” are interchangeable and refer to any vessel/container suitable for the growth of eukaryotic or prokaryotic cells, including any vessels/containers commercially available or custom-made for that purpose. In some embodiments, the phrases “tissue culture insert”, “cell culture insert”, “insert vessel”, or “transwell” refer to an apparatus designed to be placed into a tissue culture vessel for a purpose of creating multiple segregated chambers. Tissue culture vessels or vessel inserts may be constructed from materials including, but not limited to, polystyrene, a polymer, glass, plastic, etc. and may be treated/coated/constructed with a surface adapted for cell attachment. Inserts may have a porous membrane constructed from such materials as polyethylene terephthalate, polycarbonate, or any other suitable material. Surfaces of tissues culture vessels and/or inserts may be hydrophilic, hydrophobic, negatively charged, positively charged, non-ionic, or altered in texture to increase one or more surface areas. In addition, tissue culture vessels may be gas permeable and/or may include a cap/lid/closure that is gas permeable. Tissue culture vessels in accordance with embodiments include, but are not limited to, flasks, single well plates, multi-well plates, microtiter plates, bottles, Petri dishes, chamber slides, and other containers.

For the purposes of describing embodiments, the phrase “incubator” is a temperature controlled, humidified chamber with controlled carbon dioxide (CO₂) environment for maintaining cell cultures at temperatures between 30-45° C. and 1-10% CO₂.

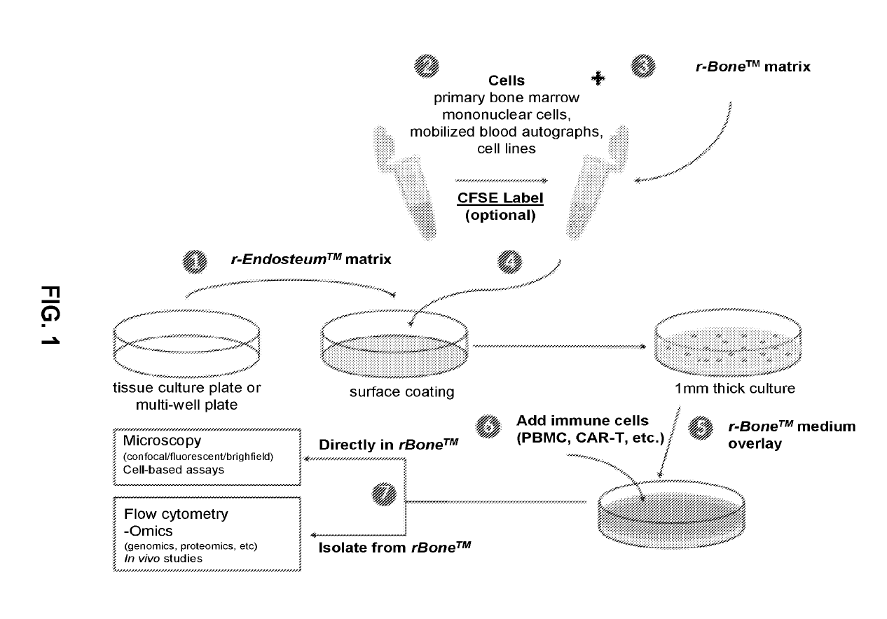

In some embodiments, a reconstructed bone marrow (rBone) is provided, which can work with an endosteum mimetic (also referred to as a reconstructed endosteum, or rEndosteum) for testing immuno-modulatory agents. The preparation and use of rBone and rEndosteum are illustrated in Example 1 and FIG. 1.

As illustrated in FIG. 1, a surface can be first coated with rEndosteum which, in one embodiment, includes fibronectin (FN) and collagen I (CI). The ratio of FN to CI, in some embodiments, is about (0.75-3.5):1, (0.5-2):1, (0.75-2):1, (0.75-1.5):1, (0.8-1.2):1, (0.9-1.1):1, or 1:1.

An rBone can be prepared by polymerizing an rBone mixture with a target cell, such as a cancer, mixed in the rBone mixture. An rBone mixture can include, in one embodiment, collagen IV (CIV), laminin (LN), fibronectin (FN), collagen I (CI), and hyaluronic acid (HA). In some embodiments, the CIV, LN, FN, VI and HA are present at a ratio of about (1.5-3):(1.5-3):(2-3):1:1, or about 2:2:2.5:1:1. The rBone mixture that contains the cell or cells can be overlaid on the surface coated with the rEndosteum, and is then allowed to polymerize (e.g., at room temperature or 37° C. In some embodiments, the rBone mixture can be placed on a surface (e.g., in a plate) without the rEndosteum coating.

An example use of the rBone is to test an immune cell's ability to impact the growth, viability or proliferation of the target cell in the rBone. In one example, a medium or growth matrix that contains the immune cell is placed in contact with the rBone (e.g., overlaid on the rBone as illustrated). Under suitable conditions, the immune cell migrates into the rBone and reacts with the target cell. A suitable condition, for instance, is a temperature from about 30° C. to about 45° C.

Example immune cells that can be tested in such a platform can be, without limitation, T cell, B cell, natural killer cell, macrophage, neutrophil, eosinophil, and dendritic cell. In one example, the immune cell is a T cell. In one embodiment, the T cell contains a chimeric antigen receptor (CAR). In another embodiment, T cell is activated by an immunomodulatory agent or is made in contact with an immunomodulatory agent in the culture medium or growth matrix. Once the T cell migrates into the rBone, the CAR on the T cell is able to bind the target cell where the CAR can exert it activity on the target cell.

In some embodiments, the culture or the biological matrix mimetic (e.g., rBone) further includes a candidate immune-modulatory agent, which may target a disease selected from an inflammatory disease or cancer. In some embodiments, the immune-modulatory agent is a checkpoint inhibitor. In some embodiments, the checkpoint inhibitor inhibits a molecule selected from the group consisting of A2AR, B7-H3, B7-H4, BTLA, CTLA-4, IDO, KIR, LAG3, PD-1, TIM-3 and VISTA.

In some embodiments, an immune activation agent can be added to the culture or the biological matrix mimetic. The addition of the immune activation agent can be before or after the immune cell migrates to the biological matrix mimetic. In some embodiments, the immune activation agent is a chemokine or a cytokine.

The impact of the immune cell on the target cell, e.g., with respect to viability and proliferation, can be observed at the platform.

Provided herein are also kits that include a biological matrix mixture, a culture medium or growth matrix, and/or an endosteum mimetic. In one embodiment, a kit further includes instructions for use. In some embodiments, each of these is placed in a compartment of a container, or in individual containers. The container may be a vial, jar, ampoule, preloaded syringe, and intravenous bag.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Reconstructed Bone Marrow (rBone)

This example demonstrates the preparation and use of a reconstructed bone marrow (rBone) for testing potential chimeric antigen receptor (CAR) T-cell therapies.

The rBone was prepared with a mixture of collagen IV (CIV), laminin (LN), fibronectin (FN), collagen I (CI), and hyaluronic acid (HA) at a ratio of 2:2:2.5:1:1. Upon adding cells to this mixture, the mixture is polymerized. In this example, the rBone was overlaid on a reconstructed endosteum (rEndosteum), which included fibronectin (FN) and collagen I (CI) at a ratio of 1:1.

This rBone platform recapitulated the comprehensive 3D microenvironment of the human bone marrow (both cellular, hematopoietic and stromal, and extracellular, soluble factors and extracellular matrix (ECM), compartments). The rBone maintained primary bone marrow cells for at least 21 days without loss of viability.

The rEndosteum matrix reconstructed human bone endosteum, connective tissue between the solid bone and bone marrow, and the rBone matrix recapitulated the extracellular matrix of human bone marrow. rBone cocktail and growth medium supplement supplied the soluble factors to complete the bone marrow microenvironment.

Procedure for Set-Up of Reconstructed Bone Marrow (rBone)

rBone matrix is thawed in a refrigerator (4° C.) overnight. Once rBone matrix has thawed, vortex briefly and place on ice for 10-15 minutes to allow air bubbles to dessipate. Changes in the color of rBone matrix indicate fluctuations in pH in response to temperature changes.

rBone matrix polymerizes at temperatures above 10° C. Keep rBone matrix refrigerated (4° C.) or on ice to avoid inadvertent polymerization. Avoid creating air bubbles while handling rBone matrix. Use pre-chilled (-20° C.) pipets and tips to avoid unwanted polymerization of the material. Use pre-warmed growth medium.

rBone cultures can be set up in various formats (i.e. 96-, 48-, 24-, and 12-well plates). Tables I, II, and III show appropriate amounts of each component.

Additional materials needed include primary cells or cell lines of interest, RPMI-1640 medium and CFSE label (optional).

With reference to FIG. 1, the following steps can be taken to set-up an rBone matrix:
1. Thaw rBone matrix. Keep rBone matrix on ice until ready to use.
2. Add rEndosteum matrix to a multi-well plate according to Table I.
3. Spread evenly to cover the entire surface of the culture vessel (Step 1 in FIG. 1).
4. Incubate for 30 minutes (or longer) at 37° C. Alternatively, plates can be pre-coated by adding rEndosteum matrix to the plate and leaving the plate at 37° C. overnight).
5. Optionally, for proliferation analysis label cells with 0.25μM CFSE prior to culture. Protect from light from this point.
6. Resuspend cells in Cell Resuspension solution according to Table I (Step 2 in FIG. 1).
7. Mix cell suspension with rBone matrix according to Table I. Place the mixture on ice (Step 3 in FIG. 1).
8. Aspirate (or pipet off) rEndosteum matrix from the plate without scratching the surface.
9. Add the cell/rBone matrix mixture to each well according to Table I (Step 4 in FIG. 1).
10. Incubate for 30-60 minutes at 37° C.
11. While rBone matrix is polymerizing, prepare rBone culture medium by adding the rBone Cocktail and rBone Supplement to 50mL of RPMI-1640 medium.
12. Overlay polymerized rBone matrix with rBone culture medium according to Table I.
13. Culture cells for the desired number of days/weeks.

TABLE I
rBone SET-UP (STANDARD PROTOCOL)

| | 96-well | | | |
|---|---|---|---|---|
| | (flat bottom) | 48-well | 24-well | 12-well |
| Approx. growth area | 0.32 | 0.95 | 1.9 | 3.8 |
| (cm²/well)^{&} | | | | |
| rEndosteum matrix | 22 | 65 | 130 | 260 |
| (μ/well) | | | | |
| # of cells/well | 1.65 × 10⁵ | 0.5 × 10⁶ | 1 × 10⁶ | 2 × 10⁶ |
| primary bone | | | | |
| marrow | | | | |
| mononuclear | | | | |
| cells | | | | |
| # of cells/well | 1.65 × 10⁴ | 5 × 10⁴ | 1 × 10⁵ | 2 × 10⁵ |
| hematopoietic | | | | |
| cell lines | | | | |
| Cell Resuspension | 3 | 10 | 20 | 40 |
| solution (μl/well) | | | | |
| rBone matrix | 33 | 100 | 200 | 400 |
| (μl/well) | | | | |
| Cell/rBone mixture | 33 | 100 | 200 | 400 |
| (μl/well) | | | | |
| rBone culture | 0.2 | 0.5 | 1 | 2 |
| medium (ml) | | | | |
| Cell Isolation | 100 | 200 | 400 | 800 |
| solution (μl/well) | | | | |

^{&}approximate growth area for Corning multi-well plates

TABLE II
CAR-T SET-UP (CAR-T on top of rBone)

| | 96-well | | | |
|---|---|---|---|---|
| | (flat bottom) | 48-well | 24-well | 12-well |
| Approx. | 0.32 | 0.95 | 1.9 | 3.8 |
| growth area | | | | |
| (cm²/well)^{&} | | | | |
| Chemoattractants | <3 | 5 | 10 | 10 |
| (μl/well) | | | | |
| CAR-T cells | 0.5-3 × 10⁵ | 1-10 × 10⁶ | 2-20 × 10⁶ | 5-50 × 10⁶ |
| (range/well) | | | | |
| rBone culture | 0.2 | 0.5 | 1 | 2 |
| medium (ml) | | | | |

^{&}approximate growth area for Corning multi-well plates

TABLE III
CAR-T SET-UP (CAR-T embedded in rBone)

| | 96-well | | | |
|---|---|---|---|---|
| | (flat bottom) | 48-well | 24-well | 12-well |
| Approx. growth area | 0.32 | 0.95 | 1.9 | 3.8 |
| (cm²/well)^{&} | | | | |
| CAR-T cells | 1-3 × 10⁵ | 0.5-1 × 10⁶ | 1-2 × 10⁶ | 2-4 × 10⁶ |
| (range/well) | | | | |
| Cell Resuspension | 3 | 10 | 20 | 40 |
| solution (μl/well) | | | | |
| rBone matrix | 33 | 100 | 200 | 400 |
| (μl/well) | | | | |
| Cell/rBone mixture | 33 | 100 | 200 | 400 |
| (μl/well) | | | | |

^{&}approximate growth area for Corning multi-well plates

The following steps can be taken for testing CAR-T cells. While rBone matrix is polymerizing, add CAR-T cells to rBone growth medium according to Table II. Gently mix CAR-T cells with rBone growth medium to evenly distribute the cells (Step 6 in FIG. 1) and overlay polymerized rBone matrix with CAR-T/rBone growth medium mixture according to Table II (Step 5 in FIG. 1). Culture cells for the desired number of days/weeks.

In the above example, the CAR-T cells are added to the growth medium. Alternatively, the CAR-T cells can be embedded in the rBone with tumor cells, as shown below:
1. Thaw rBone matrix. Keep rBone matrix on ice until ready to use.
2. Add rEndosteum matrix to a multi-well plate according to Table I.
3. Spread evenly to cover the entire surface of the culture vessel.
4. Incubate for 30 minutes (or longer) at 37° C. Alternatively, plates can be pre-coated by adding rEndosteum matrix to the plate and leaving the plate at 37° C. overnight).
5. Optionally, for proliferation analysis label cells with 0.25μM CFSE prior to culture. Protect from light from this point.
6. Resuspend CAR-T in Cell Resuspension solution according to Table III.
7. Mix the tumor cell with CAR-T cells.
8. Add rBone matrix according to Table III. Gently mix to distribute cells evenly.
9. Aspirate (or pipet off) rEndosteum matrix from the plate.
10. Add the cell/rBone matrix mixture to each well according to Table III.
11. Incubate for 30-60 minutes at 37° C.
12. While rBone matrix is polymerizing, prepare rBone culture medium by adding the rBone Cocktail and rBone Supplement to 50mL of RPMI-1640 medium.
13. Overlay polymerized rBone matrix with rBone culture medium according to Table III.
14. Culture cells for the desired number of days/weeks.

### Example 2

### Post-Culture Analysis

This example demonstrates the procedure for post-culture analysis.

For Immunostaining in rBone (Step 7, Upper Path in FIG. 1)
1. Remove rBone culture medium (**care should be taken not to disturb rBone matrix).
2. Wash rBone matrix 2-3 times with 1× phosphate buffered saline (PBS).
3. Fix cells directly in rBone matrix with 10% (v/v) neutral buffered formalin (NBF) for 15 minutes at room temperature.
4. Remove NBF and wash rBone matrix 2-3 times with 1×PBS.
** Stopping point: add PBS to prevent fixed cells from drying and store at 4° C.; complete staining and imaging within 2-3 days.
5. [optional step] Permeabilize cells with 0.1% Triton X-100 in PBS for 10 minutes at room temperature and wash rBone matrix 2-3 times with 1×PBS.
6. Block non-specific binding sites with 1% (w/v) bovine serum albumin (BSA) in PBS for 1 hour at room temperature (or at 4° C. overnight).
7. Stain with primary antibody(ies) directly conjugated to the fluorophore(s) of interest at a dilution recommended by the antibody manufacturer at 4° C. overnight (**take care to protect the cells from light).
8. Remove primary antibody solution and wash rBone matrix 2-3 times with 1×PBS.
9. [optional step] If primary antibody was not conjugated, add secondary antibody at the dilution suggested by the antibody manufacturer for 1 hour at room temperature (* *take care to protect cells from light).
10. [optional step] Remove secondary antibody solution and wash rBone matrix 2-3 times with 1×PBS.
11. Add DAPI at the dilution suggested by the manufacturer and incubate for 10 minutes at room temperature.
12. Remove DAPI solution and wash rBone matrix 2-3 times with 1×PBS.
13. Add a drop of glycerol or a commercially available fluorescence preservation solution and image within 24 hours of completion of staining.

For cell isolation (Step 7, lower path in FIG. 1)
1. Remove rBone culture medium (**care should be taken not to disturb rBone matrix).
2. Add ice-cold Cell Isolation solution to each well according to Table I.
3. Transfer the cell/matrix mixture to a microcentrifuge tube (or 15 mL conical tube).
4. Rinse the well with equal volume of the Cell Isolation solution and add to the same tube. Vigorously pipet and vortex to break-up rBone matrix and facilitate release of the cells.
5. Incubate on ice for 45-60 minutes vortexing the mixture occasionally (**if cells were labeled with CFSE, take care to protect from light). (**if at the end of 1 hour incubation time rBone matrix clumps are still visible, add one volume of Cell Isolation solution and incubate for an additional 30-60 minutes)
6. Centrifuge at 1000rpm for 7 minutes and remove supernatant.
7. Wash cells once with PBS (*this step can be skipped if dealing with very small sample sizes).
8. Continue with the analysis of interest by resuspending cells accordingly.

### Example 3

### Testing of Antibody-Drug Conjugates

Antibody-drug conjugates use antibodies to deliver a conjugated therapeutic modality to the tumor, and thus, provide a targeted delivery of a therapeutic agent to the tumor.

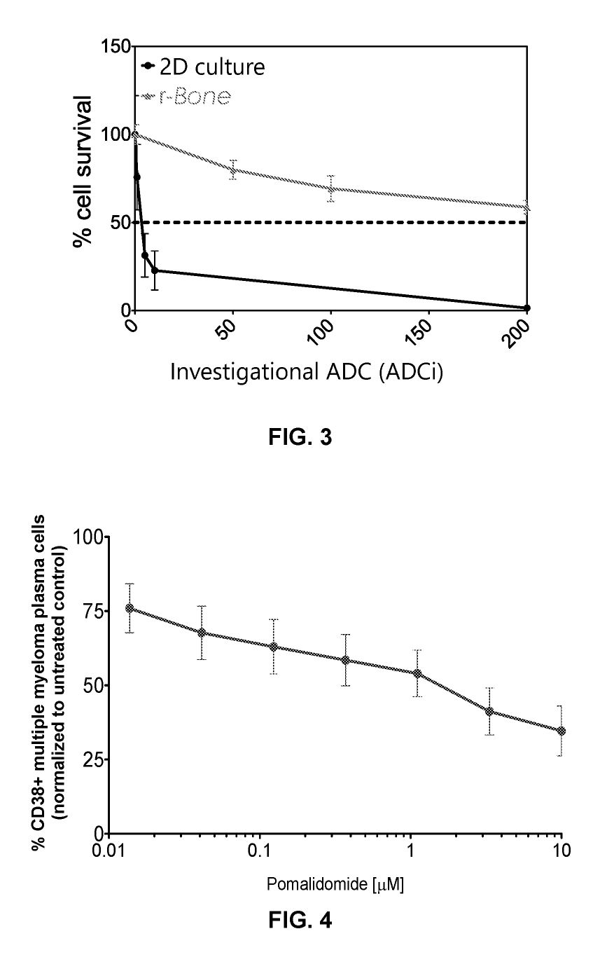

In this example, an investigational ADC was used against multiple myeloma cells cultured in standard 2D culture (lower line in FIG. 3) or in r-Bone (upper line in FIG. 3). As shown in FIG. 3, r-Bone provided a culture system complete with the major elements of human tumor microenvironment, and as such, induced drug resistance consistent with observed response in patients.

### Example 4

### Testing of Immuno-Modulatory Molecules

Pomalidomide is an immunomodulatory agent that enhances action of the immune system. It enhances T cell and natural killer (NK) cell-mediated immunity and inhibits production of pro-inflammatory cytokines (e.g., TNF-a and IL-6) that have been shown to be contribute to growth of hematopoietic malignancies.

This example tested bone marrow aspirated from patients with multiple myeloma (n=10) were cultured in r-Bone system and treated with pomalidomide. FIG. 4 shows that pomalidomide eliminated malignant plasma cells in a dose dependent manner.

### Example 5

### Testing of CAR-T

T-cells expressing a chimeric antigen receptors (CAR-T) have been shown to specifically eliminate tumor cells expressing the target molecule. This example demonstrates that the r-Bone platform can be used successfully to evaluate the efficacy of CAR-T cells.

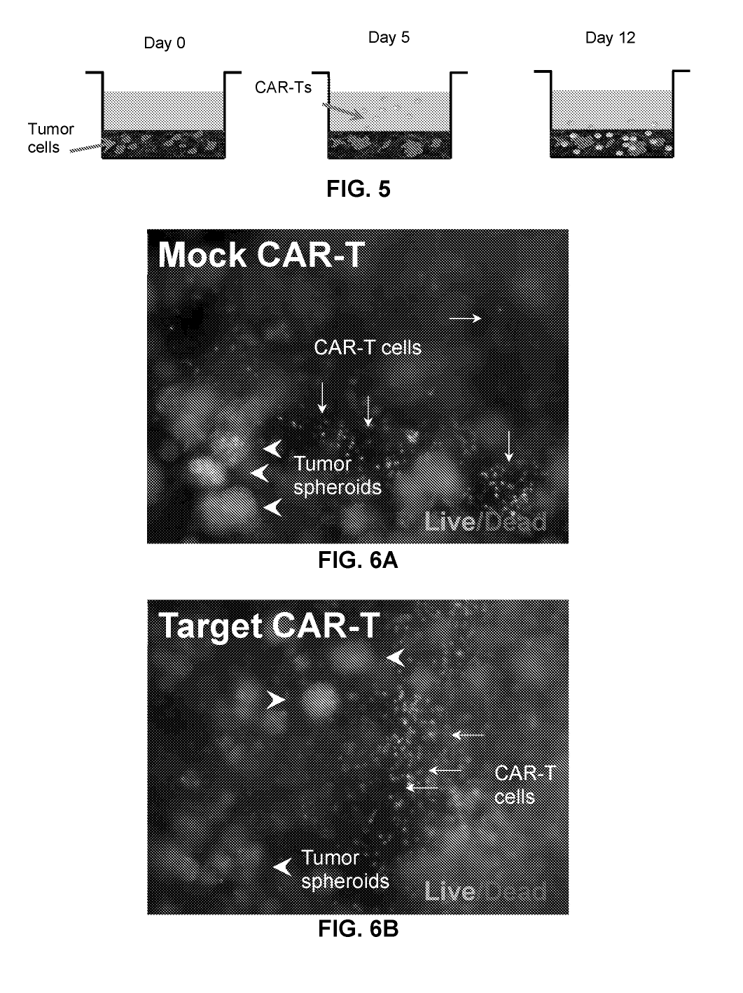

When added to r-Bone as shown in the schematic of FIG. 5, 70-90% of CAR-T cells infiltrated the ECM within 4 days. Within 2 days of ECM infiltration, target-specific CAR-T cells were activated. CAR-T cell proliferation was maintained over a period of >8 days in r-Bone culture. When added to r-Bone culture of tumor cells, target-specific CAR-Ts were found interacting with, and ultimately, killing tumor spheroids (FIG. 6A-B).

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

1. A method of preparing a platform, comprising:
contacting a) a culture comprising a medium or growth matrix and an immune cell in the medium or growth matrix, with b) a biological matrix mimetic comprising a target cell, and
allowing, under suitable conditions, the immune cell to migrate to the biological matrix mimetic.

2. The method of claim 1, further comprising preparing the biological matrix mimetic by adding the target cell to a biological matrix mixture and polymerizing the biological matrix mixture.

3. The method of claim 2, wherein the biological matrix mixture comprises collagen IV (CIV), laminin (LN), fibronectin (FN), collagen I (CI), and hyaluronic acid (HA), wherein the CIV, LN, FN, VI and HA are present at a ratio of about (1.5-3):(1.5-3):(2-3):1:1.

4. The method of claim 3, wherein the CIV, LN, FN, VI and HA are present at a ratio of about 2:2:2.5:1:1.

5. The method of claim 1, wherein the biological matrix mimetic is overlaid on a surface coated with an endosteum mimetic.

6. The method of claim 5, wherein the endosteum mimetic comprises fibronectin (FN) and collagen I (CI), and the FN and CI are present at a ratio of about (0.75-3.5):1.

7. The method of claim 1, wherein the immune cell is a T cell, B cell, a natural killer cell, a macrophage, a neutrophil, an eosinophil, or a dendritic cell.

8. The method of claim 7, wherein the T cell comprises a chimeric antigen receptor (CAR) or the T cell is activated by an immunomodulatory agent.

9. The method of claim 8, wherein the suitable conditions allow the CAR to bind the target cell.

10. The method of claim 1, wherein the culture or the biological matrix mimetic further comprises a candidate immune-modulatory agent.

11. The method of claim 10, wherein the immune-modulatory agent is a checkpoint inhibitor.

12. The method of claim 11, wherein the checkpoint inhibitor inhibits a molecule selected from the group consisting of A2AR, B7-H3, B7-H4, BTLA, CTLA-4, IDO, KIR, LAG3, PD-1, TIM-3 and VISTA.

13. The method of claim 1, further comprising adding an immune activation agent.

14. The method of claim 13, wherein the addition of the immune activation agent is before or after the immune cell migrates to the biological matrix mimetic.

15. (canceled)

16. The method of claim 1, further comprising observing an impact of the immune cell on the viability or proliferation of the target cell.

17. The method of claim 1, wherein the target cell is a tumor cell.

18. A platform, comprising:
a) a culture comprising a medium or growth matrix and an immune cell in the medium or growth matrix; and
b) a biological matrix mimetic comprising a target cell, wherein the culture and the biological matrix mimetic are in sufficient contact to allow the immune cell to migrate to the biological matrix mimetic.

19. The platform of claim 18, wherein the biological matrix mimetic is prepared by adding the target cell to a biological matrix mixture and polymerizing the biological matrix mixture.

20. The platform of claim 19, wherein the biological matrix mixture comprises collagen IV (CIV), laminin (LN), fibronectin (FN), collagen I (CI), and hyaluronic acid (HA), wherein the CIV, LN, FN, VI and HA are present at a ratio of about (1.5-3):(1.5-3):(2-3):1:1.

21. The platform of claim 20, wherein the CIV, LN, FN, VI and HA are present at a ratio of about 2:2:2.5:1:1.

22-26. (canceled)
